(19) United States
(12) Patent Application Publication
(10) Pub. No.: US 2022/0015614 A1
(43) Pub. Date: Jan. 20, 2022

(54) SYSTEMS AND METHODS FOR SYNCHRONIZED SUCTION-INJECTION ANGIOSCOPE

(71) Applicant: University of Washington, Seattle, WA (US)

(72) Inventors: Syed Faisal, Seattle, WA (US); Alberto Aliseda, Seattle, WA (US); Eric J. Seibel, Seattle, WA (US)

(21) Appl. No.: 17/311,308

(22) PCT Filed: Dec. 11, 2019
(86) PCT No.: PCT/US2019/065617

§ 371 Date: Jun. 4, 2021

Related U.S. Application Data
(60) Provisional application No. 62/778,178, filed on Dec. 11, 2018.

(51) Int. Cl. A61B 1/015; A61B 1/00; A61B 1/313; A61B 1/018; A61B 17/22

(52) U.S. Cl. CPC A61B 1/015; A61B 1/00177; A61B 1/3137; A61B 2017/22079; A61B 1/018; A61B 17/22; A61B 2562/0247; A61B 1/00043

(57) ABSTRACT

Apparatuses and methods for imaging objects in cardiovascular system are described. In one embodiment, an apparatus includes a catheter configured to traverse a blood vessel. The catheter includes: a flushing fluid inlet configured to inject a flushing fluid into the blood vessel; a flushing fluid outlet configured to evacuate the flushing fluid from the blood vessel; and an imaging module configured to image an object in the blood vessel. A first volumetric flow of the flushing fluid into the blood vessel and a second volumetric flow of the flushing fluid out of the blood vessel are synchronized to maintain a pressure in the blood vessel below a predetermined threshold.

### CROSS-REFERENCE(S) TO RELATED APPLICATION(S)

This application claims the benefit of Provisional Application No. 62/778,178, filed Dec. 11, 2018, which is incorporated herein by reference.

### BACKGROUND

Cardiovascular diseases occur within the circulatory system of the human body, such as the heart and blood vessels. Cardiovascular diseases continue to be the leading cause of death. Mortality caused by the cardiovascular diseases is estimated to keep increasing in the future.

The heart and blood vessels can be affected by numerous problems, many of which are caused by an underlying process called atherosclerosis. Atherosclerosis is characterized by thickening of the walls of the blood vessel caused by the development of plaque at the walls. This plaque build-up reduces the lumen area of the arteries, resulting in decreased blood flow. As plaque continues to develop on the walls of the artery, the vessel lumen narrows down. The plaque development may continue within the weakened artery, ultimately resulting in a complete blockage with zero or little blood flow flowing through the artery. Such a blockage is commonly referred to as a chronic total occlusion (CTO). CTOs are primarily found in coronary arteries.

It is known that inadequate blood flow deprives the heart and other organs from critical nutrients and oxygen that are required to maintain organ functions. In certain cases, a complete or near-complete obstruction of blood flow caused by the decrease in arterial lumen area can result in a heart attack (myocardial infarction) or a stroke. In practice, CTOs usually go undetected till the onset of an associated heart disease like heart failure.

A coronary angiogram is an example of a minimally invasive diagnostic procedure that can identify CTOs within blood vessels. With a coronary angiogram, a catheter is inserted into a peripheral artery (frequently the femoral artery). The catheter is then navigated to the point of interest, where an image-enhancing die is injected into the artery. Subsequently, X-ray imaging is performed to observe the presence or absence of the dye downstream of the point of injection where a blockage is suspected. In general, an absence of the die downstream of the point of inject signifies an obstruction inside the blood vessel, for example, a CTO. However, such imaging technology exposes a patient to X-ray, ionizing radiation, and to the imaging dies during a relatively long time, which may have negative side effects on the patient. Furthermore, the above-described coronary angiogram produces 2D images only.

Catheters can serve a wide range of functionalities. Catheters are primarily used for administering fluids or gases, assisting in drainage of fluids, or providing access for diagnostic or intervention instruments in patient's body. For example, cardiologists use catheters for coronary angiograms and other contrast injection techniques described above. Furthermore, catheters are used for treating cardiovascular diseases like aneurysms. Catheter design continues to grow more complex and smaller sizes, enabling the catheters to reach narrow vessels like the coronary arteries located on the heart.

Some catheters include optical channels for illuminating and imaging the target object (e.g., a CTO) for an optical observation and diagnosis. Furthermore, catheters may include tools and attachments (e.g., scalpels, suturing tools, etc.) for various procedures to be performed on the target object (also referred to as an “object” or “an object of interest”).

However, in many applications, the lumen (opening) of the blood vessel is opaque due to the presence of erythrocytes or red blood cells that limit propagation of light. This complicates optical diagnosis and treatment of the target object. Therefore, saline may be injected via one of the lumens present in the catheter to clear or push the blood away (sometimes referred to as a “saline flushing”). Saline is known to facilitate acquisition of the optical images of sections of the body by reducing the obstructions for optical analysis.

In many cases, the type of obstruction inside the blood vessel (e.g., whether the obstruction is a partially obstructing plaque or a CTO) is unknown a priori. As a result, administering saline into the space between the obstruction and the catheter may lead to pressure increase, especially so when clearing blood in the vicinity of the CTO. For blood vessels having weakened walls (e.g., due to the presence of plaque), such a pressure increase may cause complications like dislodging of the plaque or even rapturing of the blood vessel itself. Accordingly, systems and methods are needed for improved imaging and handling of the obstructions inside the blood vessels.

### SUMMARY

This summary is provided to introduce a selection of concepts in a simplified form that are further described below in the Detailed Description. This summary is not intended to identify key features of the claimed subject matter.

Briefly, the inventive technology is directed to imaging and manipulating objects in the cardiovascular system. Some examples of such objects are plaque or blood clots on the walls or in the lumen of the artery. Development of the plaque may result in a complete blockage with zero or little blood flow flowing through the blood vessel, commonly referred to as a chronic total occlusion (CTO).

In some embodiments, a catheter (also referred to as an angioscope) traverses a blood vessel (e.g., an artery or a vein). The catheter may include one or more dedicated inlets and outlets for a flushing fluid, typically a saline solution. In operation, the flushing fluid is injected into the blood vessel through a flushing inlet, and evacuated through a flushing fluid outlet. The flushing fluid at least partially removes blood in the optical path between the target object (e.g., plaque) in the blood vessel and an imaging module carried by the catheter. In different embodiments, the imaging systems may include one or more lenses, mirrors, image sensors, fiber optics, etc. Imaging of the object of the interest may start after a predefined duration of the flushing (e.g., several seconds) that is expected to sufficiently reduce blood concentration between the object of interest and the imaging module carried by the catheter.

Operation of the flushing fluid inlet and outlet may be synchronized to balance the volumetric flow into and out of the space between the catheter and the obstruction, therefore controlling the pressure increase inside the blood vessel under a predetermined pressure threshold. In some embodiments, the inflow and/or outflow of flushing fluid may be driven by dedicated pumps. In some embodiments, the pumps may drive a pulsating flow to limit a pressure rise inside the blood vessel.

In some embodiments, the catheter may be equipped with pressure sensors and/or flow sensors that communicate with a controller which controls the flow of the flushing fluid. For example, an undesirable pressure increase may be counteracted by reducing the flow of the flushing fluid through the flushing fluid inlet, by increasing the flow through the flushing fluid outlet, and/or by reducing the duration of flow pulses. In some embodiments, flow momentum (mV) of the flushing fluid may be controlled and balanced to avoid dislodging or moving the object (e.g., a CTO) inside the blood vessel. For example, flushing fluid may be driven by a solenoid bi-directional pump (fluid mover) that alternates between injecting a flushing fluid into the blood vessel and evacuating the flushing fluid from the blood vessel.

In some embodiments, the catheter may also carry work tools for manipulating the target object. Some non-exclusive examples of such work tools are scalpels, scissors, surgical tweezers, etc. Other work tools that manipulate the plaque may be used. Some non-exclusive examples of such tools are stent or coil insertion device, clot retrieval or biopsy device, atherectomy device, and optical fiber or fibers connected to lasers for ablation. The surgeon may activate work tools after reviewing images obtained by the imaging module. In some embodiments, the plaque may be removed by the tools carried by the catheter. In some embodiments, the flow of the flushing fluid in and out of the catheter may be such that the flow in/out of the catheter causes a suction into the fluid outlet that dislodges and/or evacuates the target object out of the blood vessel.

In one embodiment, an apparatus for imaging objects in cardiovascular system includes a catheter configured to traverse a blood vessel. The catheter includes a flushing fluid inlet configured to inject a flushing fluid into the blood vessel; a flushing fluid outlet configured to evacuate the flushing fluid from the blood vessel; and an imaging module configured to image an object in the blood vessel. A first volumetric flow of the flushing fluid into the blood vessel and a second volumetric flow of the flushing fluid out of the blood vessel are synchronized to maintain a pressure in the blood vessel below a predetermined threshold.

In one aspect, the flushing fluid inlet and the flushing fluid outlet are synchronized to balance a first momentum of the flushing fluid into the blood vessel with a second momentum of the flushing fluid out of the blood vessel.

In another aspect, the apparatus includes a first fluid mover configured to provide the first volumetric flow of the flushing fluid into the blood vessel, and a second fluid mover configured to provide volumetric flow of the flushing fluid out of the blood vessel.

In one aspect, the apparatus also includes: a pressure sensor operatively coupled with a space between the catheter and the object in the blood vessel; and a controller having an input operatively coupled with the pressure sensor and an output operatively coupled with first and second fluid movers.

In one aspect, the first fluid mover provides a first pulsating volumetric flow of the flushing fluid into the blood vessel and the second fluid mover provides a second pulsating volumetric flow of the flushing fluid out of the blood vessel.

In one aspect, the first fluid mover and the second fluid mover are combined in a solenoid bi-directional pump that alternates between injecting a flushing fluid into the blood vessel and evacuating the flushing fluid from the blood vessel.

In one aspect, the imaging module is configured to acquire a video clip of the object in the blood vessel.

In another aspect, the imaging module is configured to image a sidewall of the blood vessel.

In one aspect, the imaging module is supported in an imaging channel of the catheter.

In one aspect, the apparatus also includes a work tool supported in a work channel of the catheter.

In one embodiment, a method for imaging objects in cardiovascular system includes traversing a catheter within a blood vessel. The method also includes reducing a concentration of blood between the catheter and an object in the blood vessel by: injecting a flushing fluid into the blood vessel through a flushing fluid inlet of the catheter, and evacuating the flushing fluid out of the blood vessel through a flushing fluid outlet of the catheter. The flushing fluid inlet and the flushing fluid outlet are synchronized to balance a first volumetric flow of the flushing fluid into the blood vessel with a second volumetric flow of the flushing fluid out of the blood vessel. The method also includes, after injecting the flushing fluid and evacuating the flushing fluid, imaging an object in the blood vessel.

In one aspect, the method also includes balancing a first momentum of the flushing fluid into the blood vessel with a second momentum of the flushing fluid out of the blood vessel.

In another aspect, the method includes: injecting the flushing fluid is performed by a first fluid mover; and evacuating the flushing fluid is performed by a second fluid mover.

In one aspect, the first fluid mover provides a first pulsating volumetric flow of the flushing fluid into the blood vessel and the second fluid mover provide a second pulsating volumetric flow of the flushing fluid out of the blood vessel.

In another aspect, the first fluid mover and the second fluid mover are combined in a solenoid bi-directional pump that alternates between injecting a flushing fluid into the blood vessel and evacuating the flushing fluid from the blood vessel.

In one aspect, the method also includes: sensing a pressure in a space between the catheter and the object by a pressure sensor; receiving input from the pressure sensor by a controller; and controlling the first fluid mover and the second fluid mover by the controller.

In one aspect, the method also includes at least partially removing the object from the blood vessel.

In one aspect, the at least partially removing the object is performed with a work tool supported in a work channel of the catheter.

In another aspect, the at least partially removing the object includes: dislodging the object; and evacuating the object through the flushing fluid outlet.

In one aspect, the object is a plaque.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, wherein:

FIG. 1 is a schematic diagram of a catheter in operation in accordance with an embodiment of the present technology;

FIGS. 2A and 2B are a side view and an end view, respectively, of a catheter in accordance with an embodiment of the present technology;

FIGS. 3A and 3B are a side view and an end view, respectively, of a catheter in accordance with an embodiment of the present technology;

FIG. 4 is a schematic diagram of a catheter capable of viewing a side wall of the blood vessel in accordance with an embodiment of the present technology; and

FIG. 5 is a flowchart of a method for imaging and manipulating a target object in accordance with an embodiment of the present technology.

### DETAILED DESCRIPTION

While several embodiments have been illustrated and described, it will be appreciated that various changes can be made therein without departing from the spirit and scope of the claimed subject matter.

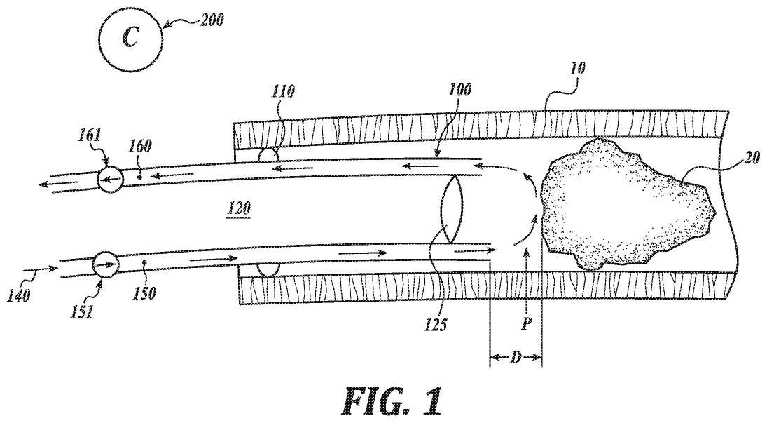

FIG. 1 is a schematic diagram of a catheter 100 in operation in accordance with an embodiment of the present technology. A retaining structure 110 keeps the illustrated catheter 100 fixed inside a blood vessel 10. A non-exclusive example of the retaining structure 110 is a pressure balloon that is inflated to press against the inner wall of the blood vessel 10, thus keeping the catheter 100 fixed in place. An obstruction 20 (e.g., plaque that may be a chronic total occlusion (CTO)) is lodged inside the blood vessel 10. In operation, the retaining structure 110 can be activated such that a prescribed distance D separates the end point of the catheter 100 from the obstruction 20 (also referred to as the target object 20). In some embodiments, such distance may extend to 15 mm or more. The catheter 100 includes an imaging channel 120 that carries an imaging module 125. The illustrated imaging module 125 is drawn as a simplified form for simplicity and clarity of representation. The person of ordinary skill would know that an imaging module may include lenses, mirrors, image sensors, optical fibers, sources of light, and/or other elements.

The catheter 100 includes a flushing fluid inlet 150 and a flushing fluid outlet 160. In operation, the flushing fluid 140 is flown into the space between the catheter 100 and the obstruction 20 through a flushing fluid inlet 150, and is evacuated through the flushing fluid outlet 160. The imaging module 125 may be activated after the flow of the flushing fluid 140 at least partially removes blood from the space around the obstruction 20. For example, the imaging module 125 may acquire still images or video clips of the obstruction 20 after about 5 seconds of flushing. Such still images or video clips may be synchronized with the flow of the flushing fluid 140. In many embodiments, the flow of the flushing fluid 140 may significantly improve images taken by the imaging module 125. For example, in some embodiments a mixture of 95% saline and 5% blood between the imaging module 125 and the obstruction 20 may already be sufficiently transparent to acquire adequate images of the obstruction 20.

In some embodiments, the incoming flow of flushing fluid 140 from the fluid inlet 150 is balanced by the outgoing flow through the flushing fluid outlet 160 such that pressure P in the space between the catheter 100 and the obstruction 20 does not increase above a certain predetermined threshold, for example, above a pressure threshold that may dislodge the obstruction 20 or rupture the blood vessel 10. In some embodiments, the flow of the flushing fluid 140 through the flushing fluid inlet 150 and the flushing fluid outlet 160 may be driven by fluid movers 151 and 161, respectively. In different embodiments, the fluid movers 151, 161 may be pumps or syringes. A controller 200 may control operation of the fluid movers 151, 161 by, for example balancing the volumetric flow and/or flow momentum (mV, where m represents mass of the fluid and V represents velocity of the fluid) out of the flushing fluid outlet of the catheter and back into the flushing fluid inlet of the catheter.

In some embodiments, the controller 200 may activate the fluid movers 151, 161 intermittently, therefore creating a pulsating flow. Without being bound to theory, it is believed that the pulsating flow may advantageously limit the pressure inside the blood vessel 10 under some use scenarios. In some embodiments, the fluid movers 151, 161 may be combined into a solenoid bi-directional pump that alternates between injecting a flushing fluid into the blood vessel and evacuating the flushing fluid from the blood vessel. In some embodiments, the fluid movers 151, 161 may also be capable of measuring the fluid flow rate.

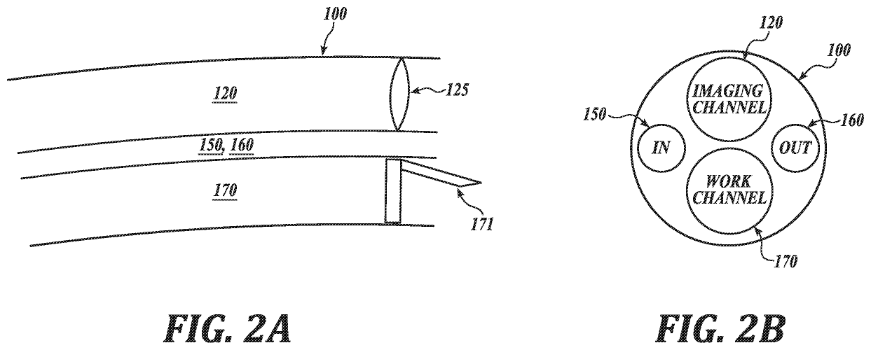

FIGS. 2A and 2B are a side view and an end view, respectively, of a catheter in accordance with an embodiment of the present technology. The illustrated catheter 100 includes a work channel 170 that houses one or more work tools (e.g., scalpel, scissors, suturing tools, or other surgical tools). In some embodiments, the surgeon may activate work tools after viewing images obtained by the imaging module 125. In different embodiments, the catheter 100 may be miniaturized down to 2.5 mm diameter or less. In the illustrated embodiment, the catheter includes one flushing fluid inlet 150 and one flushing fluid outlet 160. However, a person of ordinary skill would know that multiple flushing fluid inlets/outlets are also possible.

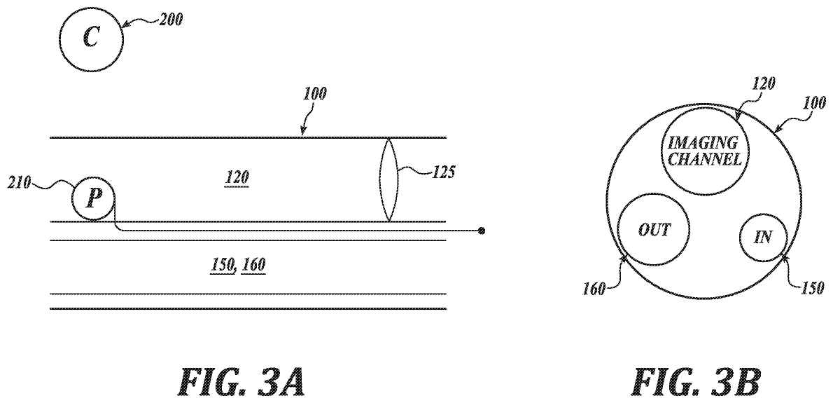

FIGS. 3A and 3B are a side view and an end view, respectively, of a catheter in accordance with an embodiment of the present technology. The illustrated catheter 100 includes a pressure sensor 210 that is configured to measure pressure in the space between the end of the catheter 100 and the target object (not shown). The controller 200 may adjust the flow through the flushing fluid inlet 150 and or flushing fluid outlet 160 based on data received from the pressure sensor 210. For example, if the pressure exceeds a predetermined threshold, the controller 200 may decrease flow of the flushing fluid through the flushing fluid inlet 150, or increase flow through the flushing fluid outlet 160, or adjust both flows through the flushing fluid inlet and flushing fluid output. As another example, the controller 200 may adjust the flow of the flushing fluid 140 such that the suction force is generated proximate to the target object by, for example, decreasing the flow of the flushing fluid through the flushing fluid inlet 150 and increasing the flow of the flushing fluid through the flushing fluid outlet 160. In some embodiments, such suction force may dislodge and evacuate the target object. The dislodging and/or evacuation of the target object may be further facilitated by the flushing fluid outlet 160 being larger than the flushing fluid inlet 150. For example, debris which could possibly clog a relatively small flushing fluid outlet may be easier removable through a larger flushing fluid outlet 160.

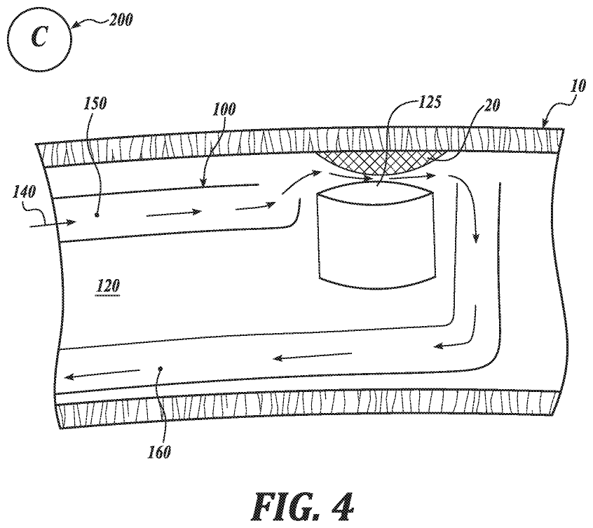

FIG. 4 is a schematic diagram of a catheter capable of viewing a side wall of the blood vessel in accordance with an embodiment of the present technology. In some applications, it may be desirable to image a side wall of the blood vessel 10, because, for example, plaque 20 may form on the side wall. In some embodiments, flow of the flushing fluid through the flushing fluid inlet 150 may be directed toward the space between the imaging module 125 and the sidewall of the blood vessel 10. In operation, the flushing fluid 140 at least partially removes blood in front of the imaging module 125, thus facilitating image taking by the imaging module 125. The mixture of the flushing fluid (e.g., saline) and blood is evacuated through the flushing fluid outlet 160. One flushing fluid inlet 150 and one flushing fluid outlet 160 are illustrated in the catheter 100, multiple flushing fluid inlets and/or flushing fluid outlets are also possible. Analogously to the embodiments described in conjunction with FIGS. 1-3A, the flow of flushing fluid may be controlled by the controller 200.

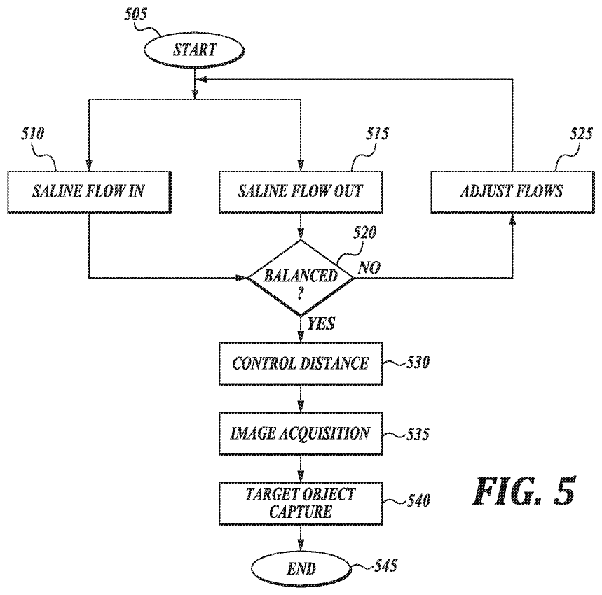

FIG. 5 is a flowchart of a method for imaging and manipulating a target object in accordance with an embodiment of the present technology. In some embodiments, the method may include additional steps or may be practiced without all steps illustrated in the flow chart.

The method starts in block 505. In block 510, the flushing fluid 140 (e.g., saline) is flown through the catheter and into the space between the imaging module and the target object. As explained above, the flushing fluid 140 removes blood between the imaging module at the target object, therefore improving visibility of the target object. In block 515, the flushing fluid or a combination of the flushing fluid and blood is removed through the flushing fluid outlet.

The flow of the flushing fluid into and out of the blood vessel is verified in block 520. For example, a balanced flow may be indicated by a relatively steady pressure in the space between the catheter and the target object. Conversely, a sudden increase in pressure in the space between the catheter and the target object may indicate excessive flow through the flushing fluid inlet. Furthermore, a decrease in pressure within the space between the catheter in the target object may indicate that the outflow through the flushing fluid outlet exceeds the inflow through the flushing fluid inlet. In some embodiments, such an imbalance of the fluid flow triggers an adjustment of the flows of the flushing fluid in block 525. Such an adjustment may be executed by, for example, controllers that are operatively coupled to the pumps or syringes that drive the flushing fluid through the flushing fluid inlet and/or outlet. In some embodiments, the controller may be operatively coupled to a solenoid bi-directional pump that alternates between injecting a flushing fluid into the blood vessel and evacuating the flushing fluid from the blood vessel.

If the flow of flushing fluid is balanced, the method may proceed to block 530 where a distance between the end of the catheter and the object of interest is controlled. In some embodiments, the operator may control the position of the catheter based on the images taken by the imaging module, followed by fixing the catheter in place by the catheter retaining structures. In some embodiments, space between the imaging module and the object of interest may be sufficiently clear within several seconds of flowing the flushing fluid through the catheter.

In block 535, image of the target object is acquired. In different embodiments, such images may be still images or video clips. An operator may rely on these images for the subsequent interventions done on the target object (e.g., plaque removal). A non-limiting example of such an intervention performed on the target object is shown in block 540, where the target object is captured. For example, the CTO may be removed either using the work tools or by dislodging/suction affected by the flushing fluid. As explained above, such dislodging/suction of the target object may be facilitated by differentially sized flushing fluid inlet and flushing fluid outlet. The method ends in block 545.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

1. An apparatus for imaging objects in cardiovascular system, the apparatus comprising:
a catheter configured to traverse a blood vessel, the catheter comprising:
a flushing fluid inlet configured to inject a flushing fluid into the blood vessel;
a flushing fluid outlet configured to evacuate the flushing fluid from the blood vessel; and
an imaging module configured to image an object in the blood vessel,
wherein a first volumetric flow of the flushing fluid into the blood vessel and a second volumetric flow of the flushing fluid out of the blood vessel are synchronized to maintain a pressure in the blood vessel below a predetermined threshold.

2. The apparatus of claim 1, wherein the flushing fluid inlet and the flushing fluid outlet are synchronized to balance a first momentum of the flushing fluid into the blood vessel with a second momentum of the flushing fluid out of the blood vessel.

3. The apparatus of claim 1, further comprising a first fluid mover configured to provide the first volumetric flow of the flushing fluid into the blood vessel, and a second fluid mover configured to provide volumetric flow of the flushing fluid out of the blood vessel.

4. The apparatus of claim 3, further comprising:
a pressure sensor operatively coupled with a space between the catheter and the object in the blood vessel; and
a controller having an input operatively coupled with the pressure sensor and an output operatively coupled with first and second fluid movers.

5. The apparatus of claim 3, wherein the first fluid mover provides a first pulsating volumetric flow of the flushing fluid into the blood vessel and the second fluid mover provides a second pulsating volumetric flow of the flushing fluid out of the blood vessel.

6. The apparatus of claim 5, wherein the first fluid mover and the second fluid mover are combined in a solenoid bi-directional pump that alternates between injecting a flushing fluid into the blood vessel and evacuating the flushing fluid from the blood vessel.

7. The apparatus of claim 1, wherein the imaging module is configured to acquire a video clip of the object in the blood vessel.

8. The apparatus of claim 1, wherein the imaging module is configured to image a sidewall of the blood vessel.

9. The apparatus of claim 1, wherein the imaging module is supported in an imaging channel of the catheter.

10. The apparatus of claim 1, further comprising a work tool supported in a work channel of the catheter.

11. A method for imaging objects in cardiovascular system, the method comprising:
traversing a catheter within a blood vessel;
reducing a concentration of blood between the catheter and an object in the blood vessel by:
injecting a flushing fluid into the blood vessel through a flushing fluid inlet of the catheter, and
evacuating the flushing fluid out of the blood vessel through a flushing fluid outlet of the catheter,
wherein the flushing fluid inlet and the flushing fluid outlet are synchronized to balance a first volumetric flow of the flushing fluid into the blood vessel with a second volumetric flow of the flushing fluid out of the blood vessel; and
after injecting the flushing fluid and evacuating the flushing fluid, imaging an object in the blood vessel.

12. The method of claim 11, further comprising:
balancing a first momentum of the flushing fluid into the blood vessel with a second momentum of the flushing fluid out of the blood vessel.

13. The method of claim 11, wherein:
injecting the flushing fluid is performed by a first fluid mover; and
evacuating the flushing fluid is performed by a second fluid mover.

14. The method of claim 13, wherein the first fluid mover provides a first pulsating volumetric flow of the flushing fluid into the blood vessel and the second fluid mover provide a second pulsating volumetric flow of the flushing fluid out of the blood vessel.

15. The method of claim 13, wherein the first fluid mover and the second fluid mover are combined in a solenoid bi-directional pump that alternates between injecting a flushing fluid into the blood vessel and evacuating the flushing fluid from the blood vessel.

16. The method of claim 11, further comprising:
sensing a pressure in a space between the catheter and the object by a pressure sensor;
receiving input from the pressure sensor by a controller; and
controlling the first fluid mover and the second fluid mover by the controller.

17. The method of claim 11, further comprising at least partially removing the object from the blood vessel.

18. The method of claim 17, wherein the at least partially removing the object is performed with a work tool supported in a work channel of the catheter.

19. The method of claim 17, wherein the at least partially removing the object comprises:
dislodging the object; and
evacuating the object through the flushing fluid outlet.

20. The method of claim 17, wherein the object is a plaque.
